(19) United States
(12) Patent Application Publication
(10) Pub. No.: US 2026/0296997 A1
(43) Pub. Date: Oct. 1, 2026

(54) METHODS FOR PRODUCING 1,1-DIFLUOROPROPENE (HFO-1252ZC) FROM VINYLIDENE FLUORIDE

(71) Applicant: Solstice Advanced Materials US, Inc., Morris Plains, NJ (US)

(72) Inventors: Vinayak Krishnamurti, East Amherst, NY (US); Colby Barrett, Charlotte, NC (US); Selma Bektesevic, Williamsville, NY (US); Joshua DeMuth, Charlotte, NC (US); Zachary Fellin, Buffalo, NY (US); Chen Liang, Charlotte, NC (US); Akbar Mahdavi-Shakib, Buffalo, NY (US); Dimitrios Papanastasiou, Buffalo, NY (US); Haiyou Wang, Amherst, NY (US)

(21) Appl. No.: 19/578,396

(22) Filed: Mar. 25, 2026

Related U.S. Application Data
(60) Provisional application No. 63/780,968, filed on Mar. 31, 2025.

(51) Int. Cl. C07C 17/281; B01J 23/745; B01J 27/053; B01J 27/128; B01J 27/138; B01J 31/02; C07C 17/383

(52) U.S. Cl. CPC C07C 17/281; B01J 23/745; B01J 27/053; B01J 27/128; B01J 27/138; B01J 31/0271; C07C 17/383

(57) ABSTRACT

A method for producing 1,1-difluoropropene (HFO-1252zc) from vinylidene fluoride (HFO-1132a) in the form of a two-step reaction is described. Step one of the reaction includes reacting HFO-1132a with an alkyl halide to produce 1,1-difluoropropane (HCFC-262fc). Step two of the reaction includes dehydrochlorination of HCFC-262fc to produce HFO-1252zc. Step one includes a catalyst, and step 2 may include a catalyst.

### CROSS-REFERENCE TO RELATED APPLICATION

This application claims priority to U.S. Provisional Application No. 63/780,968 filed Mar. 31, 2025, which is herein incorporated by reference in its entirety.

### FIELD

The present disclosure relates generally to a method for producing 1,1-difluoropropene (HFO-1252zc), and more specifically to methods for producing HFO-1252zc from vinylidene fluoride (HFO-1132a).

### BACKGROUND

Fluorocarbon fluids have properties that are desirable for use as heat transfer media, immersion coolants, liquid or gaseous dielectrics, industrial refrigerants, and other applications.

For example, 1,1-difluoropropene (HFO-1252zc) has recently found increased utility for a variety of uses. Potential end use applications of HFO-1252zc include refrigerants, either used alone or in blends with other components, solvents for organic materials, heat transfer fluid, blowing agent, and as a chemical intermediate in the synthesis of other halogenated hydrocarbon solvents.

There is a need for developing new and useful methods for synthesizing HFO-1252zc.

### SUMMARY

The present disclosure provides a method for producing 1,1-difluoropropene (HFO-1252zc) from vinylidene fluoride (HFO-1132a) in the form of a two-step reaction. Step one of the reaction comprises reacting HFO-1132a with an alkyl halide to produce 1,1-difluoropropane (HCFC-262fc). Step two of the reaction comprises dehydrochlorination of HCFC-262fc to produce HFO-1252zc. Step one comprises a catalyst, and step 2 may comprise a catalyst.

In one form thereof, the present disclosure provides a method for producing HFO-1252zc by reacting vinylidene fluoride (HFO-1132a) with an alkyl halide in the presence of a catalyst to produce a first product composition comprising 1-chloro-1,1-difluoropropane (HCFC-262fc); and reacting HCFC-262fc from the first product composition to produce a second product composition comprising 1,1-difluoropropene (HFO-1252zc).

The second reacting step is optionally conducted in the presence of a second catalyst at a second temperature, or the second reacting step comprises reacting HCFC-262fc from the first product composition in the presence of a caustic solution to produce the second product composition.

### BRIEF DESCRIPTION OF THE DRAWINGS

FIG. 1 is a schematic process flow diagram for the conversion of vinylidene fluoride to 1,1-difluoropropene (HFO-1252zc).

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “within any range encompassing any two of these values as endpoints” or “any range using any two of the foregoing values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value. For example, a range of as low as 1, 2, or 3, or as high as 8, 9, or 10 followed by this phrase encompasses ranges including 1 to 10, or 2 to 8, or 3 to 9.

As used herein, “HFO-1252zc” refers to 1,1-difluoropropene.

As used herein, “HFO-1132a” refers to 1,1-difluoroethene, or vinylidene fluoride.

As used herein, “HCC-40” refers to CH₃Cl, methyl chloride, or chloromethane.

As used herein, “HCFC-262fc” refers to 1-chloro-1,1-difluoropropane.

As used herein, the phrase “based on total moles of organic components of the composition” refers only to carbon-containing components and does not include or encompass non-carbon-containing components such as hydrogen (H₂) or hydrogen chloride (HCl).

As used herein, conversion of a reactant molecule (molecule X) during a reaction is calculated using the following equation when substantially pure reactant is used:

% conversion of molecule X=(100−molecule X mol. % in the organic components of a product mixture)

When the reactant includes impurities or recycled components, i.e., is a component in a reaction mixture, the conversion of a reactant molecule (molecule X) during a reaction is calculated using the following equations:

% conversion of molecule X=(change in X mol. %)/(X mol % in the reactant mixture)

OR

% conversion of molecule X=(X mol % in the reactant mixture−X mol % in the product mixture)/(X mol % in the reactant mixture)

As used herein, selectivity to a molecule formed during a reaction (molecule X) is calculated using the following equation:

% selectivity to molecule X=mol. % of molecule X in the organic components of a product mixture/(100−mol. % of reactant molecules in the organic components of a product mixture)×100.

### II. Overview

The present disclosure provides a method for producing 1,1-difluoropropene (HFO-1252zc) from vinylidene fluoride (HFO-1132a) according to a two-step process shown below (“Process 1”), which includes the following two steps: (i) reacting HFO-1132a with an alkyl halide, such as a methyl halide, for example methyl chloride, to produce 1-chloro-1,1-difluoropropane (HCFC-262fc), and (ii) dehydrochlorinating HCFC-262fc to produce HFO-1252zc. Other methyl halides such as methyl bromide, methyl iodide, or methyl triflate could also be used for this reaction.

Schematic equations for the two steps (“Step (i)”, “Step (ii) Vapor Phase”, and “Step (ii) Liquid Phase”) of Process 1 are represented below:

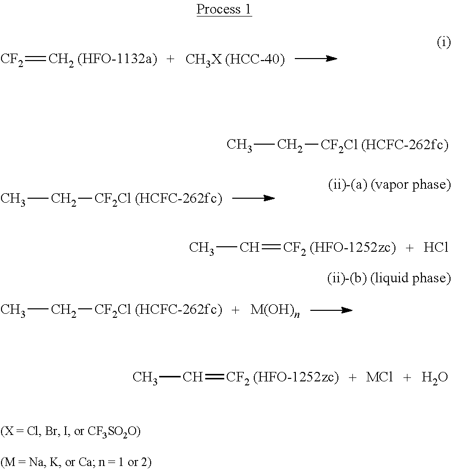

Step (i) of Process 1 may proceed through a vapor phase, liquid phase, or supercritical phase reaction, and Step (ii) of Process 1 may proceed through either a vapor phase reaction or a liquid phase reaction. Without wishing to be bound by theory, the Step (i) reaction may happen via a one-electron reduction of methyl halides as the initiation step, forming a methyl radical and a halide anion using a suitable catalyst (e.g., iron (Fe)). This methyl radical propagates the reaction via addition to the least-hindered side of the alkene employed. The resultant alkyl radical abstracts a chlorine (CI) atom from another molecule of HCC-40, with continuance of the chain reaction. A non-exhaustive list of possible byproducts include ethane, 2,2-difluorobutane, 3,3,4,4-tetrafluorohexane, 1-chloro-2,2-difluoropropane, 1,1-difluoropropan-1-ol, and 1,1-difluoropropan-2-ol. Alternatively, for example, and without wishing to be bound by theory, the Step (i) reaction may happen via carbocation formation using a Lewis acid catalyst (e.g., AlCl₃). The product of the Step (i) reaction would then be subject to dehydrochlorination conditions of Step (ii), via either a liquid phase reaction with aqueous base, or a vapor phase reaction in the presence of an appropriate catalyst.

Further details regarding each of Steps (i), (ii)-(a), and (ii)-(b) are set forth below.

### III. Process Flow

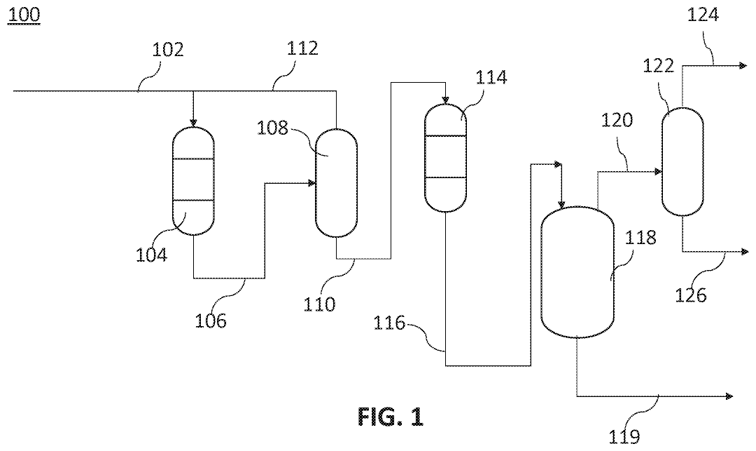

FIG. 1 is a schematic process flow diagram 100 for the conversion of vinylidene fluoride (HFO-1132a) to 1,1-difluoropropene (HFO-1252zc).

Referring to FIG. 1, stream 102 comprising nitrogen and reactant composition (e.g., HFO-1132a, methyl halide such as chloromethane (HCC-40)) for Step (i) of Process 1 enters reactor 104. Inside reactor 104, HFO-1132a undergoes Step (i) reaction to produce a first product composition comprising 1-chloro-1,1-difluoropropane (HCFC-262fc). Stream 106 including first product composition enters distillation column 108 to remove unreacted reactants from the product stream. The top stream 112 from distillation column 108 containing unreacted reactants (e.g., HFO-1132a, methyl halide such as chloromethane (HCC-40)) may be recycled to enter reactor 104 for further Step (i) reaction.

The Step (i) reaction of Process 1 is carried out in vapor phase or liquid phase in a suitable reactor, for example an autoclave reactor. The reactant (e.g., HFO-1132a, HCC-40) may be condensed into the autoclave reactor and sealed under vacuum. The contact time between the reactant composition and the reactor (and/or the catalyst in a catalytic process) may be from about 0.1 hour to about 10 hours. The process through an autoclave reactor may be a batch like process (e.g., a semi-batch process) with longer holding time compared to a tubular reactor.

The autoclave reactor may be heated to designated temperatures for the required duration. After a certain duration of contact time, the reactor may be cooled down to room temperature, and the content inside the autoclave reactor may be vacuum transferred into a cylinder. The product composition may optionally go through one or more scrubbers (not shown) to remove undesired byproducts from the reaction before being transferred into the cylinder. A gas sample may be taken from the cylinder using a gas bag (e.g., a 1 L Tedlar gas bag), and analyzed by GC/GCMS.

The reactor 104 (e.g., an autoclave reactor) can have a single wall and be heated with an external furnace or can be jacketed for heating with a circulating heat transfer medium, such as heating oil, thermal fluid, molten salt, etc. to heat at least a portion of the reactant composition during the reaction.

In an integrated process, where the product composition is not collected for analysis, the bottom stream 110 from distillation column 108 comprising HCFC-262fc enters second reactor 114 where HCFC-262fc undergoes Step (ii) dehydrochlorination reaction to produce HFO-1252zc.

The effluent stream 116 from second reactor 114 comprising HFO-1252zc, HCFC-262fc, and hydrogen chloride (HCl) gas enters caustic scrubber 118 (e.g., a KOH scrubber) for removing the HCl gas in a waste stream 119. Scrubber outlet stream 120 then goes through a dryer column (not shown) and enters a second distillation column 122 for the separation of desired product HFO-1252zc from unreacted HCFC-262fc. Top stream 124 from second distillation column 122 contains about 99.9% of desired product HFC-1252zc. Bottom stream 126 from second distillation column 122 containing HCFC-262fc is collected or recycled back to second reactor 114. Optionally, the effluent stream 116 may be sent to HCl recovery column before entering the caustic scrubber.

The Step (ii) reaction of Process 1 may be carried out in the gas or vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

The vapor phase reactor (e.g., tubular reactor 114) may be first cleaned and flushed with an inert gas such as nitrogen, followed by charging with a catalyst such as those described below. The catalyst may be pretreated within the reactor such as by drying in the manner described further below, followed by metering the reactants into the reactor to initiate the reaction.

The process flow for the Step (ii) vapor phase reaction may be in the down or up direction through reactor 114. Products may be flowed through one or more scrubbers including, for example, scrubber 118, to remove undesired byproducts from the reaction, and the reaction products may be collected by capture in a cooled vessel, for example. Optionally, the effluent stream 116 may be sent to HCl recovery column before entering the caustic scrubber.

Alternatively, the dehydrochlorination reaction of Step (ii) may be carried out in liquid phase in a suitable liquid phase reactor, for example a reactor (e.g., a Parr® reactor) made from a material which is resistant to temperature, pressure and/or corrosion such as stainless steel, nickel and its alloys, including Hastelloy (for example, Hastelloy C276), Inconel (for example Inconel 600), Incoloy, and Monel wherein the vessels which may be lined with fluoropolymers.

The liquid-phase Step (ii) reactor may be first cleaned and flushed with deionized water, followed by charging with a basic solution made from metal hydroxides (MOH) n (n=1 or 2), for example, alkali metal hydroxides (e.g., LiOH, NaOH, KOH) or alkaline earth metal hydroxides (e.g., Mg(OH)₂, Ca(OH)₂, Sr(OH)₂), or mixtures thereof, and then adding organic feed to carry out the reaction. In another embodiment, the reactor may be first cleaned, flushed with nitrogen, followed by charging with organic feed, and then adding a basic solution made from metal hydroxides (MOH)ₙ (n=1 or 2), for example, alkali metal hydroxides (e.g., LiOH, NaOH, KOH) or alkaline earth metal hydroxides (e.g., Mg(OH)₂, Ca(OH)₂, Sr(OH)₂), or mixtures thereof.

The reaction conditions for each of Step (i) and Step (ii) in Process 1 are discussed in more details below.

### IV. Step (i)

As discussed above, Step (i) of Process 1 involves reacting HFO-1132a with an methyl halide such as methyl chloride to produce 1-chloro-1,1-difluoropropane (HCFC-262fc). Without wishing to be bound by theory, the Step (i) reaction may happen via a one-electron reduction of methyl halides as the initiation step, forming a methyl radical and a chloride anion using a suitable catalyst (e.g., iron (Fe)). This methyl radical propagates the reaction via addition to the least-hindered side of the alkene employed. The resultant alkyl radical abstracts a chlorine (CI) atom from another molecule of HCC-40, with continuance of the chain reaction. The catalyst and process conditions used in Step (i) of Process 1 are detailed below.

### Step (i) Catalyst

In Step (i) of Process 1, the catalyst may comprise a metal such as an iron-based catalyst, a nickel-based catalyst, a copper-based catalyst, a palladium-based catalyst, a platinum-based catalyst, or combinations of the foregoing. For transition metal catalysts Fe, Ni, Cu, Pd, and Pt, the corresponding chloride salt may also be used as a co-catalyst. For example, Fe may be used as is, or in conjunction with FeCl₂. Similarly, Ni may be used in conjunction with NiCl₂, Pd with PdCl₂, Cu with CuCl or CuCl₂, and Pt with PtCl₂. The catalyst active to catalyze the Step (i) reaction may preferably be iron (Fe), iron chloride (FeCl₂), or a combination of Fe and FeCl₂. Suitable catalyst package active to catalyze the Step (i) reaction may optionally include tributyl phosphate (TBP) in addition to the transition metal catalysts to assist in catalytic performance.

Alternatively, in Step (i) of Process 1, the catalyst may comprise a Lewis acid catalyst including, for example, aluminum chloride (AlCl₃), aluminum chlorofluoride (AlClₓF₃₋ₓ, where x≈0.05-0.3), antimony pentafluoride (SbF₅), or combinations of the foregoing.

### Step (i) Catalyst Support

The catalyst active to catalyze the hydrogenation reaction of Step (i) includes a metal catalyst on a support. Examples of common catalyst supports include activated carbon, porous aluminosilicate typified by zeolite, aluminum oxide (alumina), silicon oxide, titanium oxide, zirconium oxide, zinc oxide, aluminum fluoride, and the like.

In the Step (i) reaction of Process 1, the transition metal catalysts described herein is supported on activated carbon, alumina, silica, aluminosilicate, or a mixture thereof. The Lewis acid catalysts including aluminum chloride, aluminum chlorofluoride, antimony pentafluoride may or may not be supported on carbon.

The supported catalyst may be produced by impregnation of any of the suitable supports with a solution of a compound of the desired metal constituent. The support may also be in the form of pellets. The size of the pellets may be as little as about 1/32 inch, about 1/16 inch, or as great as about ⅛ inch, about 3/16 inch, about ¼ inch, about 5/16 inch, about ⅜ inch, about 7/16 inch, or about ½ inch, or within any range encompassing any two of these values as endpoints, for example, from about 1/32 inch to about ½ inch, preferably from about 1/16 inch to about 3/16 inch, or more preferably about ⅛ inch. After the impregnation step, the solvent (e.g., water) may be removed using heat or under vacuum resulting in a solid mass which can be further dried and reduced to form active metal catalyst.

In the Step (i) reaction of Process 1, the catalysts supported on various catalyst supports are listed in Table 1 below.

TABLE 1
Catalysts - Step (i) Reaction of Process 1

| Catalyst | Support |
|---|---|
| Fe | carbon |
| Ni | carbon |
| Cu | carbon |
| Pd | carbon |
| Pt | carbon |
| Fe | alumina |
| Ni | alumina |
| Cu | alumina |
| Pd | alumina |
| Pt | alumina |
| Fe | silica |
| Ni | silica |
| Cu | silica |
| Pd | silica |
| Pt | silica |
| Fe | aluminosilicate |
| Ni | aluminosilicate |
| Cu | aluminosilicate |
| Pd | aluminosilicate |
| Pt | aluminosilicate |
| aluminum chloride | N/A |
| aluminum chlorofluoride | N/A |
| antimony pentafluoride | N/A |
| aluminum chloride | carbon |
| aluminum chlorofluoride | carbon |
| antimony pentafluoride | carbon |

### Catalyst Loadings

When a metal catalyst such as Ni, Cu, Pd, Pt, and/or Fe is used, its loading on a catalyst support may be as little as about 0.01 wt. %, about 0.05%, about 0.1 wt. %, about 0.15%, about 0.2 wt. %, about 0.25%, about 0.3 wt. %, about 0.4 wt. %, or as great as about 0.5 wt. %, about 1 wt. %, about 2 wt. %, about 3 wt. %, about 5 wt. %, or about 10 wt. %, based on a total weight of the catalyst and support, or within any range encompassing any two of these values as endpoints, for example, from about 0.05 wt. % to about 2 wt. %, preferably from about 0.1 wt. % to about 1 wt. %, or more preferably from about 0.2 wt. % to about 0.5 wt. %.

### Catalyst Pretreatment

For each catalyst/support combination (each row) in Table 1 used in Step (i) reaction of Process 1, the catalyst may be pretreated by a variety of methods to improve its performance and effectiveness in the reaction. For example, for the transition metal catalysts, the catalyst may be exposed to hydrogen (H₂) at elevated temperatures, for example, as low as about 100° C., about 150° C., about 200° C., about 250° C., or as high as about 300° C., about 325° C., about 350° C., or within any range encompassed by two of the foregoing values as endpoints, for example, from about 100° C. to about 350° C. Before the catalyst is exposed to hydrogen, the catalyst may optionally be calcined and/or dried at an elevated temperature, for example, as low as about 100° C., about 150° C., about 200° C., about 250° C., or as high as about 300° C., about 325° C., about 350° C., or within any range encompassed by two of the foregoing values as endpoints, for example, from about 100° C. to about 350° C.

For each catalyst/support combination in Table 1 including a Lewis acid catalyst, the catalyst may be pretreated by passing either anhydrous HF or R-22 (chlorodifluoromethane) over the catalyst at a temperature of as low as 20° C., 25° C., 30° C., 35° C., 40° C., as high as 50° C., 60° C., 70° C., 80° C., 90° C., 100° C., or within any range encompassed by two of the foregoing values as endpoints, for example, from about 20° C. to about 100° C.

### Step (i) Reaction Temperature

The reaction temperature of the Step (i) reaction may be as low as about 100° C., about 125° C., about 150° C., or as high as about 175° C., about 200° C., about 225° C., about 250° C., about 300° C., about 350° C., about 400° C. or within any range encompassed by two of the foregoing values as endpoints, such as from about 100° C. to about 400° C., or from about 125° C. to about 350° C., for example. The temperature may be preferably from about 100° C. to about 325° C., and more preferably from about 150° C. to about 300° C. The temperature range selected for the reaction may define whether the reaction occurs in a liquid phase, vapor phase, or supercritical phase. The alkyl halide reaction of Step (i) may occur in any of the liquid, vapor, or supercritical phases. The temperature for a liquid phase reaction may be from about 100° C. to about 140° C. The temperature for a supercritical phase reaction may be from about 150° C. to about 400° C., preferably from about 150° C. to about 300° C. Specific examples of additional suitable ranges are set forth below in Table 2. The numerical ranges set forth in Table 2 below are understood to be prefaced by “about”.

TABLE 2
Temperature Ranges - Step (i) Alkyl Halide Reaction

| From (° C.) | To (° C.) |
|---|---|
| 100 | 400 |
| 100 | 350 |
| 100 | 325 |
| 100 | 300 |
| 125 | 400 |
| 125 | 350 |
| 125 | 325 |
| 125 | 300 |
| 150 | 400 |
| 150 | 350 |
| 150 | 325 |
| 150 | 300 |

### Step (i) Reaction Contact Time

The contact time of the reactants with the suitable catalyst described herein of the Step (i) reaction may be as little as about 0.1 hour, about 0.5 hour, about 1.5 hours, about 2 hours, about 2.5 hours, or as long as about 3 hours, about 3.5 hours, about 4 hours, about 4.5 hours, about 5 hours, about 5.5 hours, about 6 hours, about 7, about 8, about 9, about 10, or within any range encompassed by two of the foregoing values as endpoints, such as from about 0.1 hour to about 10 hours, or from about 0.5 hour to about 9 hours. The contact time may preferably be from about 1 hour to about 8 hours, and more preferably from about 2 hours to about 6 hours.

### Step (i) Reaction Pressure

The Step (i) reaction of Process 1 may be conducted at atmospheric pressure, super-atmospheric pressure or under vacuum. For example, the Step (i) reaction of Process (i) using suitable catalyst, the pressure inside the autoclave reactor may be as little as about 100 psig, about 300 psig, about 500 psig, about 1000 psig, about 1500 psig, about 2000 psig, about 3000 psig, or within any range encompassed by two of the foregoing values as endpoints, such as from about 500 psig to about 1500 psig, or from about 100 psig to about 1200 psig. For example, the pressure may be from about 300 psig to about 2000 psig. Specific examples of additional suitable ranges are set forth below in Table 3. The numerical ranges set forth in Table 3 below are understood to be prefaced by “about”.

TABLE 3
Pressure Ranges - Step (i) Alkyl Halide Reaction

| From (psig) | To (psig) |
|---|---|
| 100 | 3000 |
| 100 | 2000 |
| 100 | 1500 |
| 100 | 1000 |
| 300 | 3000 |
| 300 | 2000 |
| 300 | 1500 |
| 300 | 1000 |
| 500 | 3000 |
| 500 | 2000 |
| 500 | 1500 |
| 500 | 1000 |

### Step (i) Reaction Conditions

Depending on the pressure and the temperature of the reaction in Step (i), the reaction may occur in a liquid phase, a vapor phase, or a supercritical phase. A summary of the preferred catalyst and temperatures as discussed above are summarized in Table 4 below. The numerical ranges set forth in Table 4 are understood to be prefaced by “about”.

TABLE 4
Summary of Preferred - Step (i) Alkyl Halide Reaction Conditions

| Catalyst | Temperature (° C.) |
|---|---|
| Fe/FeCl2/tributyl phosphate | 100-400 |
| Fe/FeCl2/tributyl phosphate | 100-350 |
| Fe/FeCl2/tributyl phosphate | 100-300 |
| Fe/FeCl2/tributyl phosphate | 100-200 |
| Fe/FeCl2/tributyl phosphate | 100-175 |
| Fe/FeCl2/tributyl phosphate | 150-400 |
| Fe/FeCl2/tributyl phosphate | 150-350 |
| Fe/FeCl2/tributyl phosphate | 150-300 |
| Fe/FeCl2/tributyl phosphate | 150-200 |

### Step (i) Conversion of the Starting Material

The Step (i) reaction of Process 1 may achieve a conversion of the starting material HFO-1132a of greater than about 10%, greater than about 20%, greater than about 30%, greater than about 40%, greater than about 50%, greater than about 60%, greater than about 70%, greater than about 80%, greater than about 90%, greater than about 95%, greater than about 99%, and for each of the foregoing, less than or equal to 100%, or within any range encompassed by two of the foregoing values as endpoints, such as from about 70% to about 100%, from about 95% to about 100%, or from about 99% to about 100%.

### Step (i) Selectivity to HCFC-262fc

The Step (i) reaction of Process 1 may achieve a selectivity to the desired product HCFC-262fc of at least about 20%, at least about 30%, at least about 40%, at least about 50%, at least about 60%, at least about 70%, at least about 80%, at least about 90%, and for each of the foregoing, less than or equal to 100%, or within any range encompassed by two of the foregoing values as endpoints, such as from about 20% to about 90%, from about 30% to about 80%, from about 40% to about 80%, or about 50% to about 80%, based on total moles of the organic components of the composition.

### V. Step (ii)-(a) Vapor Phase

As discussed above, the dehydrochlorination reaction of Step (ii) may be carried out in vapor phase in a suitable vapor phase reactor. The said vapor phase reactor may be a bundle-type tubular reactor charged with a suitable catalyst. Step (ii)-(a) of Process 1 includes dehydrochlorinating HFCC-262fc in vapor phase to produce HFO-1252zc. The catalyst and process conditions used in Step (ii)-(a) vapor phase dehydrochlorination reaction of Process 1 are detailed below.

Step (ii)-(a) Catalysts

The catalysts used for Step (ii)-(a) may include metal halides, halogenated metal oxides, neutral (or zero oxidation state) metal or metal alloy, or activated carbon in bulk or supported form. When metal halides or metal oxides catalysts are used, preferably mono-, bi-, and tri-valent metal halides, oxide and their mixtures/combinations, and more preferably mono-, and bi-valent metal halides and their mixtures/combinations. Component metals include, but are not limited to, Cr³⁺, Fe³⁺, Mg²⁺, Ca²⁺, Ni²⁺, Zn²⁺, Pd²⁺, Li⁺, Na⁺, K⁺, and Cs⁺. Component halogens include, but are not limited to, F⁻, Cl⁻, Br⁻, and I⁻. Examples of useful mono- or bi-valent metal halide include, but are not limited to, LiF, NaF, KF, CsF, MgF₂, CaF₂, CaCl₂), LiCl, NaCl, KCl, and CsCl. Halogenation treatments can include any of those known in the prior art, particularly those that employ HF, F₂, HCl, Cl₂, HBr, Br₂, HI, and I₂ as the halogenation source. When neutral, i.e., zero valent, metals, metal alloys and their mixtures are used. Useful metals include, but are not limited to, Pd, Pt, Rh, Fe, Co, Ni, Cu, Mo, Cr, Mn, and combinations of the foregoing as alloys or mixtures. The catalyst may be supported or unsupported. Useful examples of metal alloys include, but are not limited to, SS 316, Monel 400, Inconel 825, Inconel 600, and Inconel 625. Preferred catalysts include activated carbon, stainless steel (e.g. SS 316), austenitic nickel-based alloys (e.g. Inconel 625), nickel, fluorinated 10% CsCl/MgO, and 10% CsCl/MgF₂. More specifically, the Step (ii)-(a) vapor phase dehydrochlorination reaction include calcium chloride (CaCl₂)) and/or barium sulfate (BaSO₄).

Step (ii)-(a) Reaction Temperature

The temperature range for dehydrochlorination reaction of Step (ii)-(a) may be as low as about 125° C., about 150° C., about 175° C., about 200° C., about 225° C., about 250° C., or as high as about 275° C., about 300° C., about 325° C., about 350° C., about 375° C., about 400° C., about 450° C., about 500° C., or within any range encompassed by two of the foregoing values as endpoints, such as from about 125° C. to about 500° C., from about 150° C. to about 450° C., or about 175° C. to about 300° C. The temperature may be preferably from about 150° C. to about 350° C., and more preferably from about 200° C. to about 300° C. Specific examples of additional suitable ranges are set forth below in Table 5. The numerical ranges set forth in Table 5 below are understood to be prefaced by “about”.

TABLE 5
Temperature Ranges - Step (ii)-a Vapor Phase Dehydrochlorination

| From (° C.) | To (° C.) |
|---|---|
| 125 | 500 |
| 125 | 450 |
| 125 | 400 |
| 125 | 350 |
| 125 | 300 |
| 150 | 500 |
| 150 | 450 |
| 150 | 400 |
| 150 | 350 |
| 150 | 300 |
| 200 | 500 |
| 200 | 450 |
| 200 | 400 |
| 200 | 350 |
| 200 | 300 |

Step (ii)-(a) Reaction Pressure

The pressure for dehydrochlorination reaction of Step (ii)-(a) may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, about 100 psig, about 200 psig, about 300 psig, about 400 psig, about 500 psig, or within any range encompassed by two of the foregoing values as endpoints, such as from about 1 psig to about 500 psig, from about 2 psig to about 400 psig, from about 3 psig to about 300 psig, from about 4 psig to about 200 psig, from about 5 psig to about 150 psig, or from about 10 psig to about 100 psig. For example, the pressure may be from about 1 psig to about 500 psig, preferably from about 5 psig to about 300 psig, and more preferably from about 50 psig to about 150 psig. Specific examples of additional suitable ranges are set forth below in Table 6. The numerical ranges set forth in Table 6 below are understood to be prefaced by “about”.

TABLE 6
Pressure Ranges - Step (ii)-a Vapor Phase Dehydrochlorination

| From (psig) | To (psig) |
|---|---|
| 1 | 500 |
| 1 | 400 |
| 1 | 300 |
| 1 | 200 |
| 1 | 150 |
| 5 | 500 |
| 5 | 400 |
| 5 | 300 |
| 5 | 200 |
| 5 | 150 |
| 10 | 500 |
| 10 | 400 |
| 10 | 300 |
| 10 | 200 |
| 10 | 150 |
| 50 | 500 |
| 50 | 400 |
| 50 | 300 |
| 50 | 200 |
| 50 | 150 |

Step (ii)-a Reaction Conditions

A summary of the preferred catalyst and temperatures as discussed above are summarized in Table 7 below. The numerical ranges set forth in Table 7 are understood to be prefaced by “about”.

TABLE 7
Summary of Preferred - Step (ii) -a Vapor
Phase Dehydrochlorination Conditions

| Catalyst | Temperature (° C.) |
|---|---|
| CaCl₂ | 125-500 |
| CaCl₂ | 125-400 |
| CaCl₂ | 125-350 |
| CaCl₂ | 125-300 |
| CaCl₂ | 150-500 |
| CaCl₂ | 150-400 |
| CaCl₂ | 150-350 |
| CaCl₂ | 150-300 |
| CaCl₂ | 200-500 |
| CaCl₂ | 200-400 |
| CaCl₂ | 200-350 |
| CaCl₂ | 200-300 |
| CaCl₂ | 250-300 |

Step (ii)-(a) Reaction Contact Time

The contact time of the reactants with the catalyst for dehydrochlorination reaction of Step (ii)-(a) may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints, such as from about 0.1 seconds to about 120 seconds, from about 1 second to about 60 seconds, from about 5 seconds to about 50 seconds, from about 10 seconds to about 40 seconds, from about 1S seconds to about 30 seconds, or from about 20 seconds to about 25 seconds. For example, the contact time may be from about 1 second to about 60 seconds.

Step (ii)-(a) Conversion of the Starting Material

The conversion of the starting material for dehydrochlorination reaction of Step (ii)-(a) may be greater than about 10%, greater than about 20%, greater than about 30%, greater than about 40%, greater than about 50%, greater than about 60%, greater than about 70%, greater than about 80%, greater than about 90%, greater than about 95%, greater than about 99%, or within any range encompassed by two of the foregoing values as endpoints, such as from about 10% to about 99%, from about 20% to about 95%, from about 30% to about 80%, from about 40% to about 70%, or from about 50% to about 60%.

Step (ii)-(a) Selectivity to HFO-1252zc

The selectivity to the desired product HFO-1252zc for dehydrochlorination reaction of Step (ii)-(a) may be as low as about 80%, about 85%, about 89% about 90%, about 91%, about 92%, or as high as about 94%, about 95% about 96%, about 97%, about 98%, about 99%, or within any range encompassed by two of the foregoing values as endpoints, such as from about 80% to about 99%, from about 85% to about 98%, from about 89% to about 97%, from about 90% to about 96%, from about 91% to about 95%, or from about 92% to about 94%.

Step (ii)-(a) Post Processing

Referring again to FIG. 1, the product stream 116 from second reactor 116 may go through one or more post processing steps for separating desired end product HFO-1252zc with recyclable intermediates (e.g., unreacted HCFC-262fc) and/or undesired byproducts (e.g., HCl) to obtain an end product composition containing substantially pure HFO-1252zc. For example, product stream 116 may go through a caustic scrubber 118 (e.g., a KOH scrubber) to remove the HCl gas, through a dryer column to remove moisture, and then a second distillation column 122 for separating unreacted HCFC-262fc with end product HFO-1252zc. Bottom stream 126 from second distillation column 122 containing HCFC-262fc is collected or recycled back to second reactor 114. Optionally, the effluent stream 116 may be sent to HCl recovery column before entering the caustic scrubber.

The amount of desired end product HFO-1252zc in top stream 124 from second distillation column 122 may be greater than 95 mol %, greater than 98 mol %, greater than 99 mol %, or greater than 99.9 mol %, and for each of the foregoing, less than or equal to 100 mol %, for example, based on total moles of organic components in the composition.

### VI. Step (ii)-(b)

As an alternative to a vapor phase reaction, dehydrochlorination reaction of Step (ii) may also be carried out in a suitable liquid phase reactor. Step (ii)-(b) of Process 1 includes dehydrochlorinating HCFC-262fc in liquid phase to produce HFO-1252zc. The liquid phase reactants and process conditions used in Step (ii)-(b) liquid phase dehydrochlorination reaction of Process 1 is detailed below.

Step (ii)-(b) Caustic Solution

The dehydrochlorination reaction of Step (ii)-(b) may be carried out in a liquid phase reactor 114 in the presence of a caustic solution. Suitable bases that may be used to make the caustic solution include, potassium hydroxide (KOH), sodium hydroxide (NaOH), calcium oxide (CaO), lithium oxide (LiOH), and alkaline earth metal hydroxides such as calcium hydroxide (Ca(OH)₂), magnesium hydroxide (Mg(OH)₂), strontium hydroxide (Sr(OH)₂), and mixtures thereof.

The caustic solution containing feed stream in reactor 114 can be an aqueous mixture and can contain the base at any suitable strength for producing the desired reaction. For example, when KOH is used as the base, the base containing feed stream can contain base at a strength from about 5 wt. % to about 55 wt. %, preferably from about 25 wt. % to about 50 wt. %.

Step (ii)-(b) Optional Catalyst

The dehydrochlorination reaction of Step (ii)-(b) may be carried out in a liquid phase reactor 114 optionally in the presence of a phase transfer agent or catalyst for enhancing the reaction rate. Suitable phase transfer catalyst include, but are not limited to, behentrimonium chloride (C₂₅H₅₄ClN) (e.g., Aliquat®336). Other phase transfer catalysts that can be used include, but not limited to, quaternary ammonium salts, quaternary phosphonium salts, quaternary arsenium salts, sulfonium salts, crown ethers and similar molecules. In some embodiments, the optional catalyst is preferably a quaternary ammonium salt or a quaternary phosphonium salt.

Step (ii)-(b) Reaction Temperature

For a liquid phase dehydrochlorination reaction of Step (ii)-(b) of Process 1 using suitable bases as described herein, the reaction temperature of Step (ii)-(b) may be as low as about 30° C., about 35° C., about 40° C., about 45° C., about 50° C., or as high as about 100° C., about 110° C., about 120° C., about 130° C., about 140° C., about 150° C., or within any range encompassed by two of the foregoing values as endpoints, such as from about 30° C. to about 150° C., preferably from about 40° C. to about 130° C., or more preferably from about 50° C. to about 120° C., for example. Specific examples of additional suitable ranges are set forth below in Table 8. The numerical ranges set forth in Table 8 below are understood to be prefaced by “about”.

TABLE 8
Temperature Ranges - Step (ii)-b
Liquid Phase Dehydrochlorination

| From (° C.) | To (° C.) |
|---|---|
| 30 | 150 |
| 30 | 140 |
| 30 | 130 |
| 30 | 120 |
| 40 | 150 |
| 40 | 140 |
| 40 | 130 |
| 40 | 120 |
| 50 | 150 |
| 50 | 140 |
| 50 | 130 |
| 50 | 120 |

Step (ii)-b Reaction Conditions

A summary of the preferred base, catalyst, and temperatures as discussed above are summarized in Table 9 below. The numerical ranges set forth in Table 9 are understood to be prefaced by “about”.

TABLE 9
Summary of Preferred - Step (ii) -a Liquid
Phase Dehydrochlorination Conditions

| Base | Catalyst | Temperature (° C.) |
|---|---|---|
| KOH | behentrimonium chloride | 30-150 |
| KOH | behentrimonium chloride | 30-120 |
| KOH | behentrimonium chloride | 30-100 |
| KOH | behentrimonium chloride | 50-150 |
| KOH | behentrimonium chloride | 50-120 |
| KOH | behentrimonium chloride | 50-100 |
| NaOH | tetrabutylammonium bromide | 30-150 |
| NaOH | tetrabutylammonium bromide | 30-120 |
| NaOH | tetrabutylammonium bromide | 30-100 |
| NaOH | tetrabutylammonium bromide | 50-150 |
| NaOH | tetrabutylammonium bromide | 50-120 |
| NaOH | tetrabutylammonium bromide | 50-100 |

Step (ii)-(b) Conversion of the Starting Material

As demonstrated by the Examples herein, the dehydrochlorination reaction of Step (ii)-(b) may achieve a conversion of the starting material of greater than about 20%, greater than about 25%, greater than about 30%, greater than about 35%, greater than about 40%, greater than about 45%, greater than about 50%, and for each of the foregoing, less than or equal to 100%, or within any range encompassed by two of the foregoing values as endpoints, such as from about 30% to about 100%, from about 35% to about 100%, or from about 40% to about 100%.

Step (ii)-(b) Selectivity to HFO-1252zc

As demonstrated by the Examples herein, the selectivity to the desired product HFO-1252zc for the dehydrochlorination reaction of Step (ii)-(a) may be greater than about 80%, about 85%, about 90%, about 91%, about 92%, about 93%, about 94%, about 95% about 96%, about 97%, about 98%, about 99%, and for each of the foregoing, less than or equal to 100%, or within any range encompassed by two of the foregoing values as endpoints, such as from about 90% to about 100%, or from about 95% to about 100%.

### EXAMPLES

### Example 1a

### Synthesis of 1-chloro-1,1-difluoropropane (HCFC-262fc)

Example 1a shows conversion of 1,1-difluoroethene (HFO-1132a) to HCFC-262fc using chloromethane (HCC-40) and Fe/FeCl₂/tributyl phosphate catalyst in a supercritical or gas phase.

To a 300 mL Hastelloy® autoclave is charged with Iron powder (0.2 equiv, 2.2 g), FeCl₂ (0.1 equiv, 1.3 g), and tributyl phosphate (0.3 equiv, 15.9 g) under an atmosphere of N₂. The reactor is cooled using dry ice or liquid nitrogen and then evacuated, and chloromethane (0.2 mol, 10.1 g) and 1,1-difluoroethene (0.2 mol, 12.8 g) are sequentially charged. The reactor is then heated to 160° C. for 3 hours. At reaction conditions, the chloromethane and 1,1,-difluoroethene are supercritical or gaseous, and the reaction is carried out in a supercritical phase or a gas phase. The reactor is then cooled to 0° C. and unreacted 1,1-difluoroethene and chloromethane are transferred into a cylinder for recycle/reuse. The reactor may then be warmed to room temperature and the 1-chloro-1,1-difluoropropane (boiling point=25° C.) is transferred to another cylinder for product collection.

### Example 1b

### Synthesis of 1-chloro-1,1-difluoropropane (HCFC-262fc)

Example 1b shows conversion of 1,1-difluoroethene (HFO-1132a) to HCFC-262fc using chloromethane (HCC-40) and Fe/FeCl₂/tributyl phosphate catalyst in a liquid phase.

To a 300 mL Hastelloy® autoclave is charged with Iron powder (0.2 equiv, 2.2 g), FeCl₂ (0.1 equiv, 1.3 g), and tributyl phosphate (0.3 equiv, 15.9 g) under an atmosphere of N₂. The reactor is cooled using dry ice or liquid nitrogen and then evacuated, and chloromethane (0.2 mol, 10.1 g) and 1,1-difluoroethene (0.2 mol, 12.8 g) are sequentially charged. The reactor is then heated to 140° C. for 3 hours. At reaction conditions, the chloromethane is in a liquid state, and the reaction is carried out in a liquid phase. The reactor is then cooled to 0° C. and unreacted 1,1-difluoroethene and chloromethane are transferred into a cylinder for recycle/reuse. The reactor may then be warmed to room temperature and the 1-chloro-1,1-difluoropropane (boiling point=25° C.) is transferred to another cylinder for product collection.

### Example 2

### Synthesis of 1,1-difluoropropene (HFO-1252zc)

Example 2 shows conversion of HCFC-262fc to HFO-1252zc using CaCl₂) catalyst.

A tubular reactor with a heated zone 4.5 inches long is packed with pelletized CaCl₂). Through this catalyst bed, 1-chloro-1,1-difluoropropane is introduced at a gas flow rate of 135 sccm at a temperature of 260° C. The effluents from the reactor are comprised of a mixture of 1-chloro-1,1-difluoropropane, the target compound HFO-1252zc and hydrogen chloride gas which may be removed via a caustic scrubber. The target compound is purified by distillation.

### Example 3

### Dehydrochlorination of 1-chloro-1,1-difluoropropane (HCFC-262fc) using KOH caustic solution

A 1-liter Parr reactor equipped with agitator is used to convert HCFC-262fc to HFO-1252zc. 15 g of Aliquat 336 is added to reactor. 224 g of 99.9% HCFC-262fc and 370 g of 45% KOH are added to reactor. Reactor temperature of reaction is slowly increased to 60° C. The reaction is run for 6 hours at 60° C. After reaction, the organic content is vented off to a chilled product collection cylinder. GC analysis shows the organic content contains 65 mol % of HFO-1252zc and 33% of HCFC-262fc.

### Example 4

### Dehydrochlorination of 1-chloro-1,1-difluoropropane (HCFC-262fc) using KOH caustic solution

A 500 mL round bottom flask equipped with a magnetic stir bar is fitted with a condenser set to 0° C., an addition funnel, and a thermocouple. The outlet of the condenser is fitted to a dry ice-cooled trap. 200 mL of an aqueous solution of NaOH (20-50 wt %) is charged to the flask, followed by the addition of tetrabutylammonium bromide (1-10 wt %). The solution is heated to 70° C. At this temperature, HCFC-262fc is added dropwise. The desired product HFO-1252zc is evolved from the solution as a gas and collected in the cold trap.

### ASPECTS

Aspect 1 is a method for producing 1,1-difluoropropene (HFO-1252zc), comprising: reacting vinylidene fluoride (HFO-1132a) with an alkyl halide in the presence of a catalyst to produce a first product composition comprising 1-chloro-1,1-difluoropropane (HCFC-262fc); and reacting HCFC-262fc from the first product composition to produce a second product composition comprising 1,1-difluoropropene (HFO-1252zc).

Aspect 2 is the method of Aspect 1, wherein the first reacting step is conducted at a temperature of from about 100° C. to about 300° C.

Aspect 3 is the method of Aspects 1 or 2, wherein the methyl halide comprises chloromethane (HCC-40).

Aspect 4 is the method of any one of Aspects 1 to 3, wherein the catalyst is an iron-based catalyst.

Aspect 5 is the method of any one of Aspects 1 to 4, wherein the catalyst is selected from iron, iron chloride, and a combination thereof.

Aspect 6 is the method of any one of Aspects 1 to 5, wherein the catalyst package further comprises tributyl phosphate.

Aspect 7 is the method of any one of Aspects 1 to 6, wherein the first reacting step is conducted in vapor phase, liquid phase, or supercritical phase.

Aspect 8 is the method of any one of Aspects 1 to 7, wherein the first reacting step comprises contacting the vinylidene fluoride (HFO-1132a) and the methyl halide with the catalyst for a duration of from about 0.1 hour to about 10 hours.

Aspect 9 is the method of Aspect 1, further comprising: distilling the first product composition to produce a distilled composition comprising at least 90 mol % HCFC-262fc.

Aspect 10 is the method of any one of Aspects 1 to 9, wherein the second reacting step is conducted in vapor phase in a tubular reactor.

Aspect 11 is the method of Aspect 10, wherein the second reacting step is conducted in the presence of a second catalyst at a second temperature.

Aspect 12 is the method of Aspect 11, wherein the second catalyst is calcium chloride.

Aspect 13 is the method of Aspect 11, wherein the second catalyst is barium sulfate.

Aspect 14 is the method of any one of Aspects 11 to 13, wherein the second temperature is from about 125° C. to about 500° C.

Aspect 15 is the method of any one of Aspects 11 to 14, further comprising: distilling the second product composition to produce an end product composition comprising at least 99 mol % HFO-1252zc.

Aspect 16 is the method of any one of Aspects 1 to 9, wherein the second reacting step comprises reacting HCFC-262fc from the first product composition in the presence of a caustic solution to produce the second product composition.

Aspect 17 is the method of Aspect 16, wherein the caustic solution is formed from a base comprising at least one of KOH, NaOH, CaO, Ca(OH)₂, Mg(OH)₂, Sr(OH)₂, and combinations of the foregoing.

Aspect 18 is the method of Aspects 16 or 17, wherein the second reacting step is conducted at a second temperature of from about 30° C. to about 150° C.

Aspect 19 is the method of any one of Aspects 16 to 18, wherein the second reacting step is conducted in the presence of a phase transfer catalyst.

Aspect 20 is the method of Aspect 19, wherein the phase transfer catalyst comprises a salt such as (but not limited to) a quaternary ammonium salt (e.g., behentrimonium chloride) or a quaternary phosphonium salt (e.g., hexadecyltributylphosphonium bromide).

Aspect 21 is the method of any one of Aspects 16 to 20, further comprising: distilling the second product composition to produce an end product composition comprising at least 99 mol % HFO-1252zc. Aspect 22 a product composition comprising HFO-1252zc made according to any one of Aspects 1 to 21.

What is claimed is:

1. A method for producing 1,1-difluoropropene (HFO-1252zc), comprising:
reacting vinylidene fluoride (HFO-1132a) with an alkyl halide in the presence of a catalyst to produce a first product composition comprising 1-chloro-1,1-difluoropropane (HCFC-262fc); and
reacting HCFC-262fc from the first product composition to produce a second product composition comprising 1,1-difluoropropene (HFO-1252zc).

2. The method of claim 1, wherein the first reacting step is conducted at a temperature of from about 100° C. to about 300° C.

3. The method of claim 1, wherein the alkyl halide comprises chloromethane (HCC-40).

4. The method of claim 1, wherein the catalyst is an iron-based catalyst selected from iron, iron chloride, and a combination thereof.

5. The method of claim 1, wherein the first reacting step is conducted in vapor phase, liquid phase, or supercritical phase.

6. The method of claim 1, wherein the first reacting step comprises contacting the vinylidene fluoride (HFO-1132a) and the methyl halide with the catalyst for a duration of from about 0.1 hour to about 10 hours.

7. The method of claim 1, further comprising:
distilling the first product composition to produce a distilled composition comprising at least 90 mol % HCFC-262fc.

8. The method of claim 1, wherein the second reacting step is conducted in vapor phase in a tubular reactor.

9. The method of claim 8, wherein the second reacting step is conducted in the presence of a second catalyst at a second temperature.

10. The method of claim 9, wherein the second catalyst is calcium chloride.

11. The method of claim 9, wherein the second catalyst is barium sulfate.

12. The method of claim 9, wherein the second temperature is from about 125° C. to about 500° C.

13. The method of claim 9, further comprising:
distilling the second product composition to produce an end product composition comprising at least 99 mol % HFO-1252zc.

14. The method of claim 1, wherein the second reacting step comprises reacting HCFC-262fc from the first product composition in the presence of a caustic solution to produce the second product composition.

15. The method of claim 14, wherein the caustic solution is formed from a base comprising at least one of KOH, NaOH, CaO, Ca(OH)₂, Mg(OH)₂, Sr(OH)₂, and combinations of the foregoing.

16. The method of claim 14, wherein the second reacting step is conducted at a second temperature of from about 30° C. to about 150° C.

17. The method of claim 14, wherein the second reacting step is conducted in the presence of a phase transfer catalyst.

18. The method of claim 17, wherein the phase transfer catalyst comprises a salt selected from a quaternary ammonium salt and/or a quaternary phosphonium salt.

19. The method of claim 14, further comprising:
distilling the second product composition to produce an end product composition comprising at least 99 mol % HFO-1252zc.

20. A product composition made according to the method of claim 1.
